(19) United States
(12) Patent Application Publication
(10) Pub. No.: US 2004/0247589 A1
(43) Pub. Date: Dec. 9, 2004

(54) Anti-tumor activity from reptile serum

(76) Inventors: Ofer Binah, Nofit (IL); Aaron Ciechanover, Haifa (IL); Gila Maor, Kiriat Motzkin (IL)

Correspondence Address: WINSTON & STRAWN, PATENT DEPARTMENT, 1400 L STREET, N.W., WASHINGTON, DC, 20005-3502, US

(21) Appl. No.: 10/761,528

(22) Filed: Jan. 20, 2004

(30) Foreign Application Priority Data
Jul. 19, 2001 (IL) IL 144447

(51) Int. Cl. A61K 39/395; C07K 16/00

(52) U.S. Cl. 424/130.1; 530/388.1

(57) ABSTRACT

The invention discloses anti-tumor activity in the sera of certain reptiles, more particularly in the serum of alligators or crocodiles. The active principle resides in one or more serum proteins or polypeptides that inhibit the growth of mammalian tumor cells in culture while being practically devoid of such inhibitory activity toward normal cells in culture. The active fractions obtained from the sera show no overt toxicity in healthy mammals, and this novel anti-tumor agent may therefore be used for the prevention, investigation, treatment or diagnosis of neoplasms.

### CROSS-REFERENCE TO RELATED APPLICATIONS

This application is a continuation of International application PCT/IL02/00590 filed Jul. 18, 2002, the entire content of which is expressly incorporated herein by reference thereto.

### FIELD OF THE INVENTION

The invention relates to anti-cancer agents derived from serum of certain reptiles, to processes for producing same and for the use of these agents in the prevention, treatment, investigation or diagnosis of cancer.

### BACKGROUND OF THE INVENTION

The presence of anti-tumor factors in the serum of mammals has been described in numerous publications in the scientific literature. By way of example, an activity described as tumor necrosis factor, abbreviated as TNF, was detected in the serum of certain mammals including rodents (Cancer Letters 6, 235-240, 1979). This activity was found to be induced by the injection of bacterial endotoxins such as lipopolysaccharide (LPS), or by infection of animals with bacteria that produce such endotoxins. Additional purification and characterization of this activity has disclosed that there are different subtypes of TNF activity, one of the most common forms being referred to as TNF alpha.

Tumor necrosis factor (TNFα) is a pleiotropic cytokine which has been implicated in immunological and inflammatory responses as well as in pathogenesis of endotoxic and septic shock (Tracey and Lowry, The role of cytokine mediators in septic shock. Adv. Surg. 23, 21-56, 1990). TNFα is one of several cytokines released mainly by mononuclear phagocytic cells in response to various stimuli, including bacterial infection and probably also viral, fungal or parasitic infections.

Further disclosures involve the isolation and characterization of different polypeptides possessing anti-tumor activity from the sera of mammals including humans, as disclosed for example in U.S. Pat. No. 4,309,418. A human secreted glycoprotein having antitumor activity is disclosed in WO 90/10651.

Antibodies and their fragments are widely used for therapy and diagnosis of cancers. For example, U.S. Pat. No. 5,169,774 discloses monoclonal anti-human breast cancer antibodies, while U.S. Pat. No. 6,136,311 discloses methods for treatment and diagnosis of cancer using monoclonal antibodies. Methods for imaging and treating bladder cancer using antigen-specific antibody is are disclosed in international application WO 00/12761.

Several forms of recombinant antibody fragments can be designed to substitute for large intact immunoglobulin molecules. These options include Fab fragments or Fv fragments that are stabilized and/or covalently linked utilizing various strategies (Bird et. al., Science 242, 423-426, 1988).

Small fragments of antibodies are advantageous for pharmaceutical applications for cancer targeting and imaging for example when small antigen binding molecules are needed to penetrate into large solid tumors.

International patent application WO 98/17301 discloses peptides derived from shark immunoglobulins for inhibiting retroviruses and for inhibiting growth of tumor cells. The peptide preparations are useful for inhibiting diseases associated with retroviral infection, such as acquired immunodeficiency syndrome. The peptides also inhibit growth of tumor cells, especially sarcomas and leukemias.

Nowhere is it taught or suggested in the background art that anti-tumor activity may be found in the serum of reptiles, or more specifically in the serum of alligators or crocodiles.

### SUMMARY OF THE INVENTION

The present invention is directed to anti-tumor agents obtained from the serum of certain reptiles. More particularly, the present invention is directed to an anti-tumor agent derived from the serum of alligators or crocodiles.

The present invention relates to an agent or agents that are polypeptides found in the serum of normal healthy alligators, characterized in that they show specific anti-tumor activity.

The present invention provides anti-tumor agents derived from the serum of reptiles, comprising at least one serum protein from the serum of normal reptiles.

According to the present invention, the disclosed agents are able to discriminate between normal proliferating cells and tumor (malignant) cells. This remarkable feature distinguishes the agents of the present invention from common chemotherapeutic agents.

In one preferred embodiment, the anti-tumor agents of the present invention are immunoglobulin molecules. More preferred molecules according to the present invention are active fragments or domains derived from same immunoglobulin molecules, while additional preferred compounds are peptides derived from the binding sites of such immunoglobulin fragments or domains obtained from reptiles sera.

The present invention further relates to a process for recovering the activity of the anti-rumor agents in comparatively enriched form by fractionation of alligator serum.

In one embodiment, the enrichment process comprises the steps of: precipitating proteinaceous material from the serum by partial saturation of the serum with ammonium sulfate; re-dissolving the precipitate and desalting the recovered proteins by dialysis or other suitable means; and fractionating the recovered proteinaceous material by gel filtration, size exclusion chromatography, ion exchange chromatography or the like.

In yet another embodiment the present invention relates to additional processes enabling purifying the active polypeptides, determining at least part of their amino acid sequence, and characterizing any active domain or domains or specific peptide fragments. Such active fragments and peptides are also within the scope of the invention, as are peptide derivatives of any such active domain.

In another aspect the present invention provides methods of using the anti-tumor agents for the investigation, prevention, treatment and diagnosis of tumors in mammals.

In one embodiment, two or more individual polypeptides from the sera of healthy alligators or fragments or peptides derived therefrom, may be used as a mixture having enhanced anti-tumor activity. The anti-tumor agents may be used on their own or covalently coupled to known anticancer drugs in order to enhance the specificity of the latter, or to a detectable marker in order to facilitate location of tumors.

The present invention is explained in greater detail in the description, Figures and claims below.

### BRIEF DESCRIPTION OF THE FIGURES

FIGS. 1A and 1B: Effect of alligator serum, denoted Serum Y, on [³H]-Thymidine incorporation of PN71 (A) and EL4 (B) tumor cell lines. [³H]-Thymidine incorporation in presence of FCS is defined as 100%.

FIG. 2: Morphological damage to PN71 cells induced by alligator serum, denoted Serum Y. After 2 hr. incubation with Serum Y, the majority of cells were lysed, and isolated nuclei are observed.

FIGS. 3A and 3B: Dose-response relationship of effect of alligator serum, denoted Serum Y, on proliferation of murine tumor cell lines (A), and normal cells (B).

FIG. 4: Effect of alligator serum on human tumor cells. Cells were exposed to alligator serum for 18 hours prior to assaying their metabolic (MTT) activity.

FIG. 5: Photographs illustrating the anti-tumor effect of alligator serum, denoted Serum E on T47D cells (ductal breast carcinoma).

FIG. 6: Photographs illustrating the anti-tumor effect of alligator serum, denoted Serum Y, on HeLa cells (epithelial cervix carcinoma).

FIG. 7: Lack of effect of alligator serum on normal human cells. The negative value indicates a slight stimulatory action on peripheral blood lymphocytes.

FIG. 8: Effect of fraction Ya and the chemotherapy drug Cytosar on proliferation (BrdU assay) of normal human bone marrow cells and murine leukemia PN71 cells. 100% inhibition is defined for normal bone marrow cells in presence of 2% Cytosar.

FIG. 9: Heat inactivation of anti-tumor activity of alligator serum. The anti-tumor activity was assayed in PN71 cells.

FIG. 10: Effect of dithiotreitol (DTT) on anti-tumor activity of alligator serum, denoted Serum Y. The anti-tumor activity was assayed in PN71 cells.

FIG. 11: Effect of dialysis of alligator serum, denoted Serum Y, on its anti-tumor activity in PN71 cells.

FIG. 12: Distribution of alligator serum (Serum Y) anti-rumor activity against PN71 cells, in the precipitate (Ya) and in the supernatant (Yb) components obtained from 45% ammonium sulfate fractionation procedure.

FIG. 13: Distribution of the alligator serum (Serum Y) anti-tumor activity against EL4 cells, in the precipitate (Ya) and the supernatant (Yb) components obtained from 45% ammonium sulfate fractionation procedure. The effect of ammonium sulfate fractionation of horse serum is provided for the sake of comparison.

FIG. 14: A representative FPLC fractionation of the 45% ammonium sulfate precipitate obtained from alligator serum. The figure depicts the protein content distribution throughout the column.

FIG. 15: Anti-tumor activity expressed as % inhibition of MTT activity in PN71 cells, of the FPLC protein fractions of FIG. 14.

FIG. 16: Dose-response relationship of the active fractions (No. 12-13) derived from the FPLC fractionation shown in FIG. 15, and fraction Ya (the crude precipitate), compared to unfractionated alligator serum (Serum Y).

FIG. 17: SDS PAGE of various fractions (3-20) eluted from FPLC column chromatography. Every two consecutive fractions were combined to one sample for gel electrophoresis.

FIG. 18: SDS-PAGE analysis of denatured and non-denatured fractions of FIG. 14 that demonstrate anti-tumor activity as shown in FIG. 15.

FIG. 19: Effects of distinct FPLC fractions obtained from alligator serum on the morphology of PN71 cells. The effect of ammonium sulfate precipitate (Fraction Ya) is shown for comparison.

FIG. 20: Synergistic anti-tumor effects of distinct FPLC fractions obtained from alligator serum on the metabolic activity of PN71 tumor cells. The effect of ammonium sulfate precipitate (Fraction Ya) is shown for comparison.

FIG. 21: Dose-response curves of fractions 11-14, obtained from the FPLC fractionation of fraction Ya monitored in the absence or presence of 2 μl of fractions 3+4, AF1. Response is defined as % inhibition of MTT activity in PN71 cells.

FIG. 22: Effect of antibodies against anti-tumor molecules on anti-tumor activity of fraction Ya as measured by inhibition of MTT activity in PN71 cells.

FIG. 23: In vivo anti-tumor activity of alligator serum (denoted Serum Y). The figure depicts the in vivo anti-tumor effect of fraction Ya in EL4-bearing mice.

### DETAILED DESCRIPTION OF THE INVENTION

The present invention discloses that the blood of normal healthy reptiles contains a unique activity of an anti-tumor agent.

The present invention further discloses that the agents accountable for that activity can be substantially enriched and even obtained in isolated fractions from the serum of certain alligators.

According to one aspect, the present invention relates to the anti tumor activity of reptile serum in the treatment, diagnosis or investigation of cancer. More particularly, the present invention relates to the anti tumor activity of alligator or crocodile serum.

The anti-tumor activity of the sera obtained as described herein below can be demonstrated by exposing rumor cells in culture to varying concentrations of the complete serum without any further manipulation. As will be exemplified herein below, the anti-tumor activity is present when alligator serum is added to cultures of tumor cells or cultured rumor cell lines from a wide range of murine or human rumors. Notably, no such anti-tumor activity was demonstrable when using the sera obtained from healthy individuals of several mammalian species that served as control sera.

The same alligator sera, when added to normal cells in culture or normal cell lines, are practically devoid of any significant toxicity. Furthermore, the sera, or more preferably, active fractions derived from it, when injected into healthy mice show no overt toxicity.

The anti-tumor activity of alligator serum was demonstrated on a battery of human tumor cell lines. The following exemplary human tumor cell lines were killed by alligator serum: 1) T47D, ductal breast carcinoma; 2) HeLa, epithelial cervix carcinoma; 3) U937, human histiocytic lymphoma cells; 4) Saos-2, osteosarcoma.

According to another aspect, the present invention provides methods for recovery of this activity from the serum of alligators.

According to one embodiment, blood obtained from alligators is collected, preferably under aseptic conditions. Blood is allowed to coagulate and the serum fraction is separated from the clot, though it is possible also to work with the plasma fraction of blood which is prevented from coagulating by the addition of various anti-coagulants as are well known in the art. Sera samples may be stored until use in sterile containers at −70° C. The sera from individual animals may be pooled or may be used separately.

According to yet another embodiment, the anti tumor agent or agents are obtained from the serum of normal alligators by a process comprising the steps of fractionating the serum by the addition of ammonium sulfate salt in an amount of about 45% of the amount necessary to form a saturated solution; centrifuging the serum to recover the precipitated proteins; re-dissolving the precipitate and desalting the recovered proteins; gel filtering the desalted proteins; and collecting at least one active fraction.

Fractionation of reptile serum such as alligator serum is carried out in order to enrich the anti-tumor activity present in the unfractionated samples. The anti-tumor activity may be used as an enriched fraction obtained from such sera. Most preferably, it will be desirable to isolate the active component or components, and to utilize them in essentially pure form.

The active compound in alligator serum is a proteinaceous material. It can be precipitated by ammonium sulfate, is inactivated by elevated temperature (56° C.) or by agents which reduce disulfide (S—S) bonds of polypeptides, including but not limited to Dithiotreitol (DTT), or digested by proteolytic enzymes as are known in the art.

It will be appreciated by the artisan that many equivalent procedures may be utilized to provide enriched serum fractions containing substantially enriched anti-tumor activity in terms of units of activity per unit of protein.

More preferably the active component or components of alligator serum will be isolated and used in essentially pure form. The activity may be obtained using suitable isolation procedures as a single protein or polypeptide, or as a defined mixture of synergistically acting polypeptides.

In yet another aspect, the present invention relates to antibodies raised against active components of alligator serum, said antibodies are shown herein below to inhibit the anti-tumor activity.

In one embodiment, such antibodies will be used in the process of purifying the active components by purification methods known in the art.

In yet another embodiment, the antibodies may be used to detect the existence of the active components in body fluids and when bound to tumor cells in vitro or in vivo in diagnostic procedures.

The purified polypeptides so isolated will be sequenced at least in part. Furthermore, defined polypeptide domains or peptide regions which retain the anti-tumor activity of the intact protein may advantageously be used as well.

According to a further aspect, the anti-tumor agents of the present invention, used for the prevention, investigation, treatment and diagnosis of tumors, may be administered to a mammal by any suitable route of administration. The anti tumor agents may be administered as a full serum, serum fractions, partially purified components, isolated components, active fragments, peptides or derivatives thereof. Pharmaceutical and diagnostic compositions comprising as an active ingredient one or more anti-tumor agents derived form alligator serum may further comprise any pharmaceutically acceptable diluents or excipients. As will be exemplified hereinbelow, these active components may also act synergistically.

Depending on the tumor type to be treated, the anti-tumor effect of alligator serum or its fractions may be induced within 2-3 hours after exposure to the serum. As will be exemplified hereinbelow using test cultures of tumor cells, as early as 30 min after addition of alligator serum to the culture medium, large clumps of cells are observed. The aggregation inducing activity and the lethal activity of the serum may reside in separate protein fractions of the serum. As will be exemplified herein, these separable activities may be used synergistically.

Based on size exclusion chromatography it appears that the anti-tumor activity resides in a protein or proteins having a molecular weight of approximately 150,000 Daltons under native conditions. The aggregation inducing activity, elutes from such columns in a fraction having an estimated molecular weight of at least 200,000 Daltons (e.g. about 700,000 Daltons). It will be appreciated by the skilled artisan that these are merely estimates that will be refined upon further purification of the active proteins.

Under denaturing or reducing conditions the anti-tumor activity can be recovered as a pair of polypeptides having molecular weights of approximately 60,000 Daltons and 30,000 Daltons. This suggests that the anti-tumor activity resides in a protein comprising at least two subunits of polypeptide chains. It is to be understood that these are the major protein bands in the fractions having the anti-tumor activity of the alligator serum. Nevertheless, the artisan will appreciate that the activity may be dependent wholly or in part on a minor protein component of these fractions.

The results suggest that a novel mechanism or mechanisms of action may be involved in the anti-tumor activity of alligator serum. Irrespective of the mechanism of action, the anti-tumor activity of alligator serum is useful for the prevention or treatment of tumors in vivo, for the investigation of tumors in vivo and in vitro, and for the diagnosis or imaging of tumors in vivo and in vitro. The isolated proteins, and/or active fragments thereof having the anti-tumor activity, will be useful as pharmaceutical compositions, as diagnostic reagents, and as imaging agents.

According to one embodiment, the isolated proteins or active fragments thereof will further be useful as carriers for known anti-tumor drugs, to enhance their specificity or as targeting molecules to enhance their delivery to the tumor cells. The chemical reactions necessary to bind a given anticancer drug to the alligator serum derived anti-tumor protein or peptide are well known in the art, and may conveniently utilize coupling to a free amine (e.g., an ε-amine of a lysine residue, the α-amine of the N-terminus, etc.), coupling to a free carboxyl (e.g., a carboxyl of an aspartic or glutamic acid residue, or the carboxyl of the C-terminus) or coupling to any other suitable reactive group on one of the side chains of the amino acids comprising the sequence of said protein or peptide (e.g., the hydroxyl of serine or tyrosine residues, the sulfhydryl of cysteine residues, etc.).

According to yet further embodiment, the protein or protein fragment that is to be used as a diagnostic reagent or as an imaging agent will be coupled to any suitable marker as is known in the art. The marker may binds directly, via a covalent bond, to the agent, or through a chelator. Any appropriate linker or spacer may connect the agent to the marker or to the chelator. The marker may comprise an atom with a nucleus suitable for magnetic resonance imaging (MRI); a radioactive atom suitable for radiolabelling, including but not limited to gamma emitters suitable for detection on X-ray film, gamma emitters for single photon emission computed tomography (SPECT), or other detection means, positron emitters suitable for positron emission tomography (PET); and any other suitable chemical marker. The tumor imaging agent may also comprise an anti-tumor protein or peptide coupled to a contrast agent suitable for computerized tomography (CT). Radioactive derivatives may also be used to perform diagnostic tests based on radioimmunoassay. Alternatively, enzyme linked immunoassays can be performed for diagnostic purposes utilizing non-radioactive derivatives of the anti-tumor agents. For in vitro tests it is also possible to use fluorescent derivatives obtained by covalently coupling the anti-tumor agent to a fluorescent chromophore. One skilled in the art will appreciate the many variations and modifications of the reagents that are possible to provide a derivative suitable for imaging or diagnostic purposes, using chemical derivatives that include coupling to any reactive group in the amino acid sequence.

The invention will better be understood by reference to the following examples. The skilled artisan will appreciate that the following examples are merely illustrative and serve as non limitative exemplification of the principles of the present invention and that many variations and modifications are possible within the scope of the currently claimed invention as defined by the claims which follow.

### EXAMPLES

Materials and Methods

Cell Lines and Culture Conditions

Murine Tumor Cells:

1. PN71, CTL hybridoma.

2. EL4, leukemia.

3. NSO, myeloma (not secreting immunoglobulins).

4. 4T00.1, myeloma.

Human Tumor Cells:

1. T47D, ductal breast carcinoma.

2. HeLa, epithelial cervix carcinoma.

3. U937, histiocytic lymphoma.

4. Saos-2, osteosarcoma.

Normal Murine and Rat Cells:

1. Rat fetal primary fibroblasts.

2. Rat neocortical pyramidal neurons (2 day old).

3. Murine cartilaginous growth center of a neonatal (2 day old) mandibular condyle.

Normal Human Cells:

1. Fallopian tube-derived cells (FTDC); mixed population of secretory, ciliary and fibroblast-like cells.

2. Peripheral blood lymphocytes.

Cell Preparation and Culture Conditions

Murine PN71 and EL4 cell lines were grown in DMEM medium (high glucose, 2 mM L-glutamine, 1 mM Na-Pyruvate, 10% FCS, Penicillin/Streptomycin). Human HeLa and Saos-2 and murine NSO and 4TOO.1 tumor cells were grown in DMEM medium (high glucose, 2 mM L-glutamine, 10% FCS, Penicillin/Streptomycin). Human T47D cells were grown in RPMI (10% FCS, insulin, Penicillin/Streptomycin). U937 cell were grown in RPMI (10% FCS, 2 mM glutamine, Penicillin/Streptomycin). Rat primary fibroblasts were prepared by trypsinization of 16-18 day old rat fetuses. Fibroblasts were groan in Weymouth medium, containing 10% FCS, Penicillin/Streptomycin. Fallopian tube derived cells were obtained from total abdominal hysterectomy (unrelated to cancer). The tissue was cut and washed under sterile conditions in DMEM/F12. Subsequently, the epithelial cell layer was gently scrapped into a sterile tube. Red blood cells were spun down by a mild centrifugation. Epithelial cells were then cultured in 96 multi-well dish at a density of 2×10⁵ cells/ml for 5 days before treatment with alligator serum or other treatments. The culture medium contained DMEM/F12, 10% FCS, 2 mM L-glutamine, Penicillin/Streptomycin. Mandibular condyles were aseptically dissected out of 2-day old mice, cleaned of all soft tissues and cultured under conditions that favor its endochondral ossification and a normal reaction towards various external factors. Subsequently, condyles were transferred onto collagen sponges (U257, 16DS Prolex Princeton N.J.) placed on stainless steel grids and fed with BGJb medium (Fiton Jackson modification, Beit Haemek, Israel) supplemented with 2% FCS, 200 μg/ml ascorbic acid, streptomycin and penicillin.

Preparation of Alligator Serum

Blood was obtained from normal alligators (Alligator mississipiensis) when sacrificed for their hide, and collected (from each individual separately) in sterile plastic tubes. Blood was then allowed to coagulate for 24 hr. at 4° C. Thereafter, the serum fraction was separated from the clot by centrifugation (20 min., 4000 rpm, 4° C.), though it is possible also to work with the plasma fraction of blood which is prevented from coagulating by the addition of various anti-coagulants as are well known in the art. Sera were stored separately from individual animals at −70° C. In the following experiments, serum samples from individual animals were tested separately.

Treatment with Alligator Serum

Cells Grown in Suspension

Cell cultures (2×10⁵ cells/ml) and mandibular condyles (2 organs/ml), were incubated for 18 hours with various concentrations (0.5-5% v/v) of Y serum. Control cultures were incubated with the following non-heat-inactivated sera. Fetal calf serum (FCS, in most experiments), horse serum (HS) or rabbit serum (RS). At the end of incubation period, cells/organs were thoroughly washed with fresh medium and assayed.

Cells Attached to the Dish

Prior to the assay, the fibroblast-like cells which were grown to confluence on the dish, were peeled off using trypsin-EDTA solution (0.25% trypsin, 0.05% EDTA in Puck's saline A, Beit-Haemek, Israel) for 2-7 min. at 37° C. Subsequently, cells were washed with fresh medium and diluted to 2×10⁵ cells/ml. Cells were allowed to attach to the plastic dish for at least 6 hrs. prior to treatment with alligator serum.

Assays of the Anti-Tumor Activity of Alligator Serum

The following methods were utilized to assay the anti-tumor activity of alligator serum, and fractions thereof.

MTT Activity

The enzymatic activity of the mitochondrial dehydrogenases is considered as a reliable test for cell vitality. Briefly, 100 μl of cells (2×10⁵/ml) were incubated in 96 multi-well dish, in the presence of either alligator serum or control serum. At the end of the incubation period, 25 μl of 5 mg/ml MTT solution (Sigma) were added for 2 hours at 37° C. The formazan dye crystals were dissolved in 100 μl 0.4 N HCl/isopropanol, added to each well. The color intensity was measured in ELISA reader at 570 nm.

[³H]-Thymidine Incorporation

At the end of the incubation period, 5 μCi/ml of [³H]-methyl Thymidine (5.0 Ci/mmol, Negev Nuclear Center, Beer-Sheva, Israel) were added for 2 hours. Cells were then washed once with PBS and twice with cold 5% TCA (+1 mM Thymidine) and lysed with 0.1% SDS in 0.1% N NaOH. DNA (macromolecules) was precipitated by ice cold 5% TCA (final concentration), and collected on nitrocellulose membrane (0.45 μm pore size) using a vacuum apparatus. Membranes were washed twice with 5% TCA, dried and counted in standard toluene-based scintillation fluid.

Cell Proliferation Assay—BrdU

Cell proliferation was assayed using the commercial ELISA kit BIOTRACK™ (RPN250, Amersham Life Sciences). The assay is based on incorporation of 5-bromo-2′-deoxyuridine (BrdU) during DNA synthesis. After treatment of cells with the tested agent for 24 hours, cells are incubated for 18 hours in the presence of 10 μM BrdU. This pyrimidine analog is incorporated into DNA and is detected using specific antibody conjugated to peroxidase following 3,3′,5′,5′-tetramethylbenzidine (TMB) as a substrate. The absorbance of the enzymatic product is measured at 450 nm.

Histological Procedures

PN71 cells (serum-treated and non-treated) were pelleted and fixed with 3% glutaraldehyde (in cacodylate buffer) and 1% OsO₄. Cells were then dehydrated in graduated alcohols and embedded in Epon. 3 μm sections were cut and stained with 1% toluidine blue.

Fractionation of Alligator Serum

Precipitation in Ammonium Sulfate

Ammonium sulfate (pure crystals) was added to alligator serum to 45% saturation final concentration. After 30 minutes on ice, precipitate was separated by centrifugation (15 min., 13,000 rpm). Sediment was resuspended in 20 mM HEPES+150 mM NaCl and fractionated again in 45% ammonium sulfate. The final sediment was collected in half of the original volume of HEPES/NaCl buffer (see above). Proteins in the supernatant (1st one) were separated in 80% (final concentration) of ammonium sulfate and resuspended in half volume of HEPES/NaCl buffer. Both supernatant (denoted as fraction Nb below) and precipitate (denoted as fraction Ya below) fractions were dialyzed against 1000 volumes of the same HEPES/NaCl buffer for 3×1 hour in 6.4 cm Dialysis membrane tubing (MWCO 12-14K, Spectrum Medical Industries Inc., Texas), having a cutoff of 12,000-14,000 Daltons. The dialysates were tested for biological activity as described above.

FPLC (Fast Performance Liquid Chromatography) Fractionation

2 ml (˜70 mg protein) of Ya (sediment fraction of 45% ammonium sulfate fractionation) were loaded on FPLC column (S-200, 120 ml). Serum proteins were eluted in 18 fractions of 2.4 ml each. Every 2 adjacent fractions were joined and concentrated (using Centricon 10, cat No 4205, Amicon) to about 450 μl and assayed for biological activity (MTT) and content of proteins (Bradford reaction).

Bradford Method for Protein Determination

20 μl of protein-containing samples (1-20 μg) were reacted with 180 μl of Bradford reagent (0.01% Coomasie Brilliant Blue, 5% ethanol, 10% phosphoric acid). The developed color was determined between 5-30 min. and read in 600 nm in ELISA-reader.

Antibodies Against Anti-Tumor Active Components in Alligator Serum

Immunization: six BALB/C mice were injected subcutaneously 4 times. 2-3 weeks apart, with 50 μg of Active Fraction II (AF2) derived from Serum Y. Three weeks after the last injection, sera were screened for the presence of neutralizing monoclonal antibodies (mAb).

Fusion: three days before fusion, mice were boosted with 300 μg of AF2 intraperitoneally. PEG-induced fusion of spleen cells with murine myeloma cells (NSO), and selection for hybridoma cells were performed according to routine procedures.

Hybridoma cells were grown in RPMI supplemented with 5% NCTC (MA Biqproducts), 1% NEM, 10% DCCM-1 and 10% FCS. Cells were maintained in suspension at 37° C. in an atmosphere of 10% CO₂.

Cloning of positive hybridoma cells: positive colonies (based on the neutralization assay) were cloned by the limiting dilution technique. Clones demonstrating anti-Ya activity were subsequently subcloned in soft agar.

Neutralization assay: one hundred μl of serum taken from immunized mice or positive clones-derived conditioned media, were pre-incubated for 60 min at 37° C., with 10 μl of fraction Ya (Fraction Ya is the precipitate fraction obtained by treatment of Serum Y with 45% ammonium sulfate). This Ya volume is capable of killing over 90% of PN71 leukemia cells (a CTL hybridoma cell line). Subsequently, 50 μl of PN71 cells (4×10⁵ cells/ml) were added for additional 18 hrs incubation period. Thereafter, the MTT activity (representing cell viability) of PN71 cells was determined, as described herein above.

Definition of a mAb unit: One “unit” of mAb activity was defined as the amount of serum or medium that neutralizes >60% of Ya killing capacity of PN71 cells, as determined by the MTT assay.

Characterization of the Clones:

(1) Identification of immunoglobulin classes: Typing and subtyping analysis of the mAb was performed in flat-bottomed immunoplates (Immunoplate 1, Nunc, Denmark) coated with rabbit anti-mouse γ1/K, γ1, γ2a, γ2, γ3, c, a, A (Southern Biotechnologies Association, Birmingham, Ala., USA) for 2-4 hrs at room temperature. Free binding sites were blocked by incubating the wells overnight at 4° C. with 1% BSA in PBS. Subsequently, the plates were washed with 0.05% Tween-20-phosphate buffer saline (PBS). Undiluted media were added to the wells, and incubated at room temperature for 2-4 hrs. The wells were then washed and alkaline phosphatase conjugated to the respective subclass antibodies was added and incubated at room temperature for 2 hrs. The substrate p-nitrophenyl phosphate was added in 0.05 M bicarbonate buffer (pH 9.8). Optical density is read at 405 nm by means of an ELISA reader.

(2) Quantification of mAb concentration was accomplished by ELISA using standard purified mouse IgG1 (PharMingen) at known concentration.

(3) The biological activity of the clones was determined using the neutralization assay, as described above.

(4) Specific activity: Each clone was characterized by its specific activity, defined in units of activity per ng antibody, as described herein above.

Experimental Results

Anti-Tumor Activity of Alligator Serum in Murine Tumor Cell Lines

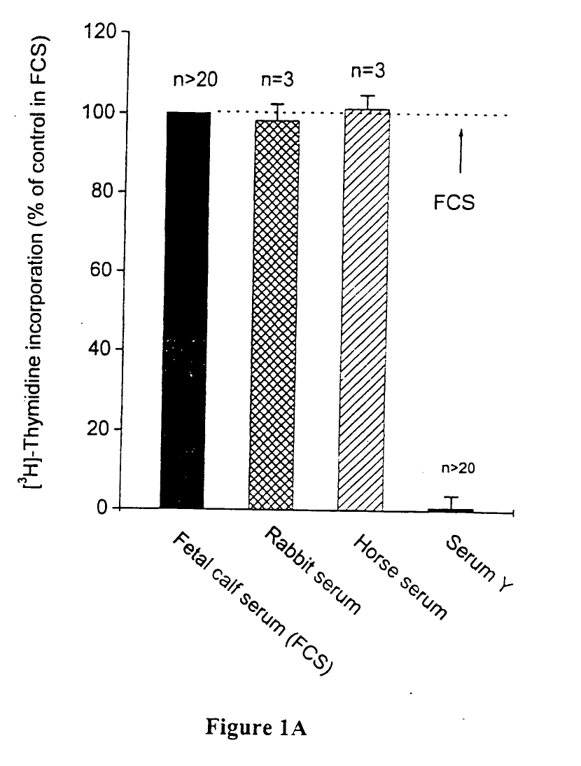

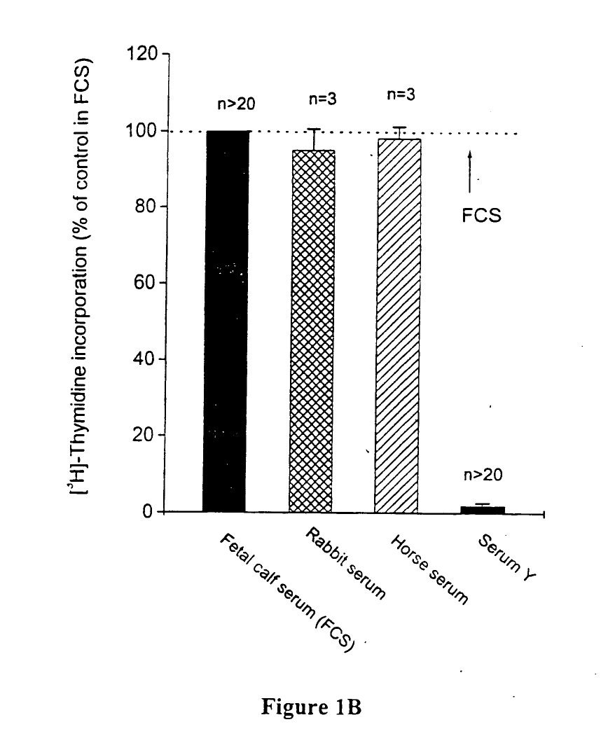

FIG. 1 demonstrates that alligator serum, denoted Serum Y in the figures, at a concentration of 5%, caused complete inhibition of proliferation of two murine tumor cell lines: PN71 (panel A) and EL4 cells (panel B). Cells were exposed to alligator serum (or other sera) for 18 hours prior to addition of [³H]-Thymidine. The [³H]-Thymidine incorporation in fetal calf serum (FCS) was defined as 100%. Results are expressed as mean±SEM. Addition of 5% rabbit serum or horse serum to the culture medium (containing FCS) had no effect on tumor cells proliferation.

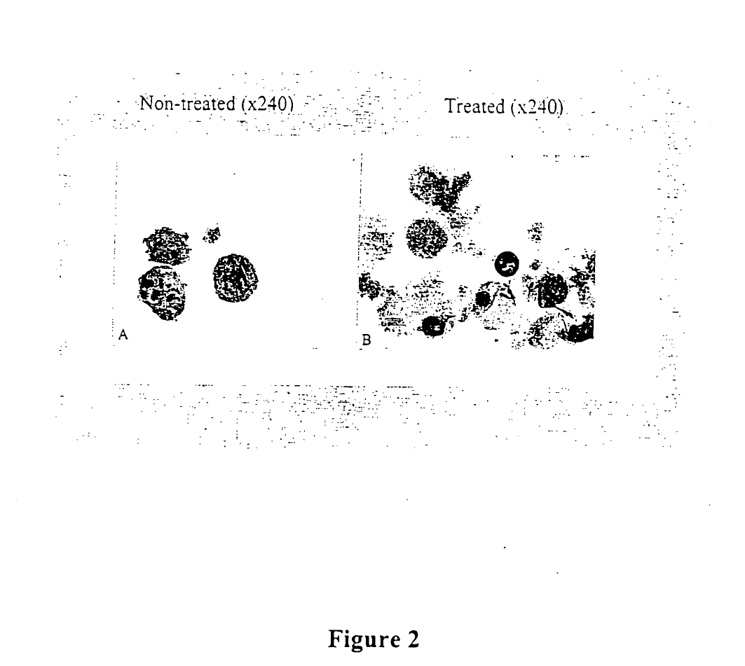

FIG. 2 demonstrates that 2 hours after incubation with alligator serum, denoted Serum Y, the majority of cells were lysed, and isolated nuclei are observed.

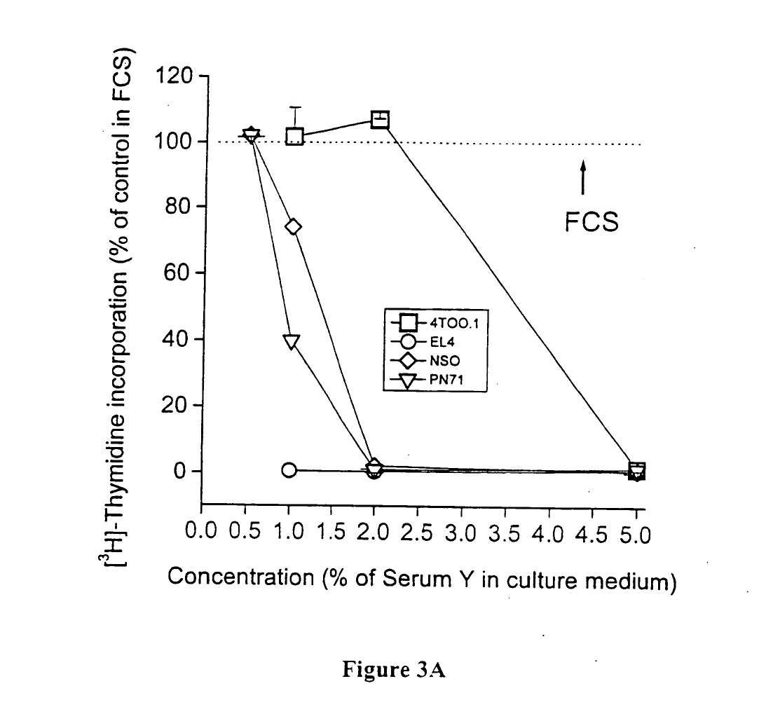

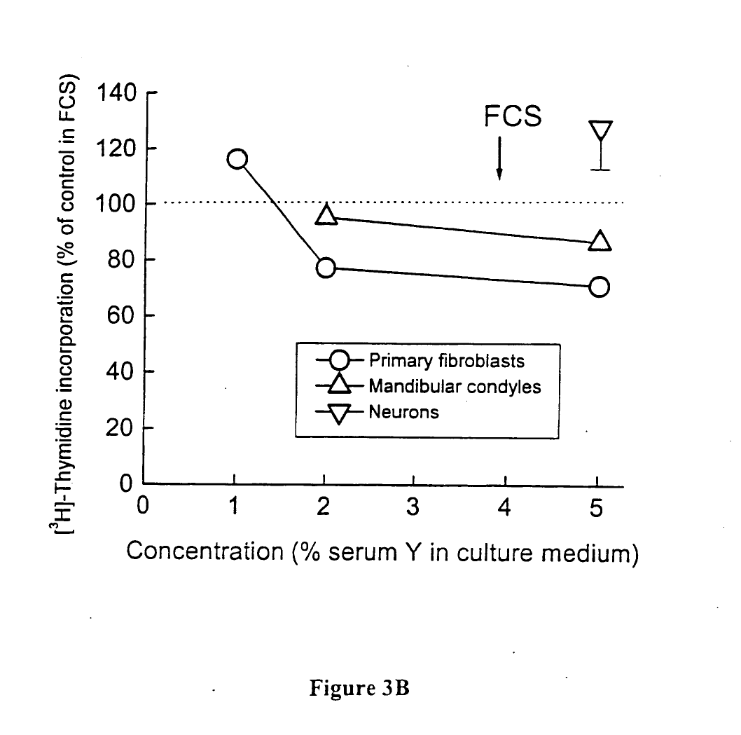

Dose-response relationships of the anti-tumor activity of alligator serum were generated in 4 tumor cell lines (FIG. 3A, n=3-4 experiments in each cell type) Cells were exposed to alligator serum (or other sera) for 18 hours prior to addition of [³H]-Thymidine. [³H]-Thymidine incorporation in fetal calf serum (FCS) was defined as 100%. Results are expressed as mean±SEM.

At the concentration range studied (0.5 to 5%), alligator serum affected 4TOO.1, NSO and PN71 in a dose-dependent manner, while ELA cells were already maximally inhibited by 1% alligator serum. To explore alligator serum toxicity, we tested its effect in two normal rat cell types and in a murine organ culture: primary fetal fibroblasts and neocortical pyramidal neurons, and fetal condyles, respectively (FIG. 3B, n=2-3 experiments in each cell type). The condyle organ culture is an important control, because it represents an experimental model of differentiation as well as of proliferation. Altogether, while condyles and neurons were minimally affected, 5% alligator serum (completely destroying tumor cells), caused only a 20% inhibition of primary fibroblasts proliferation.

Anti-Tumor Activity of Alligator Serum in Human Tumor Cell Lines

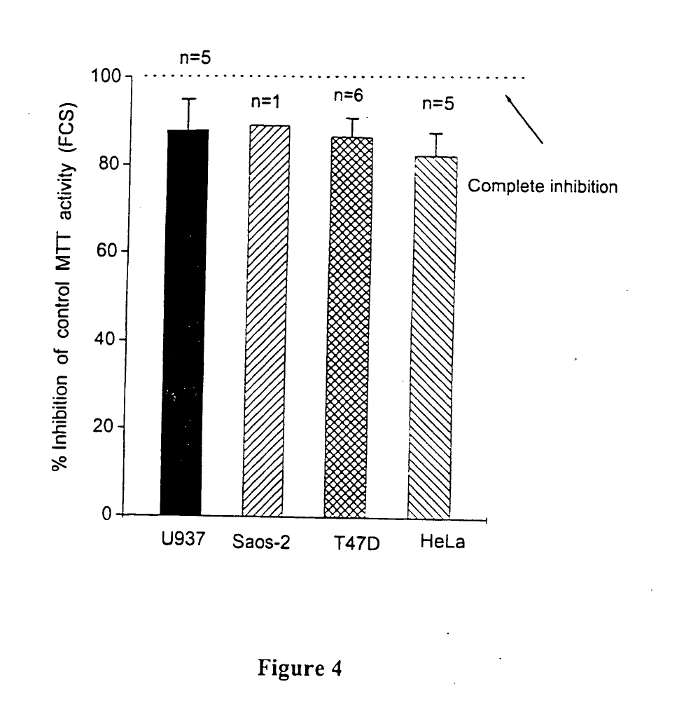

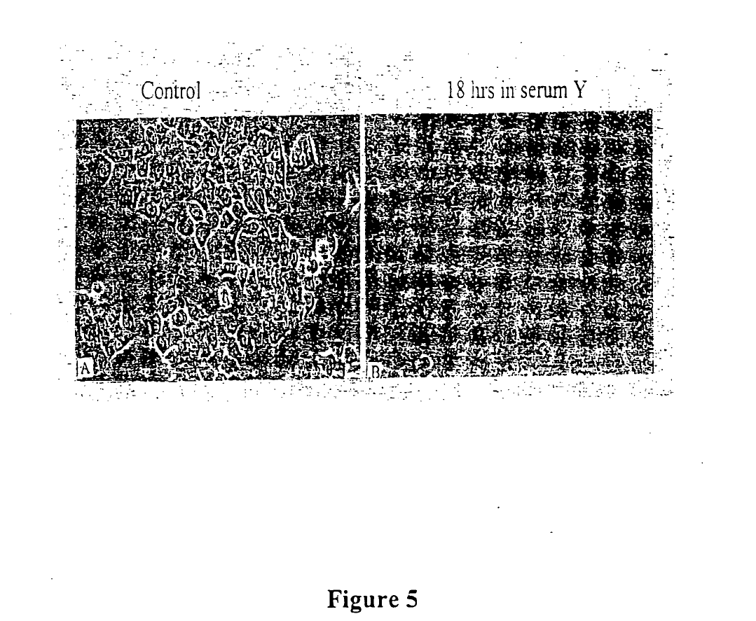

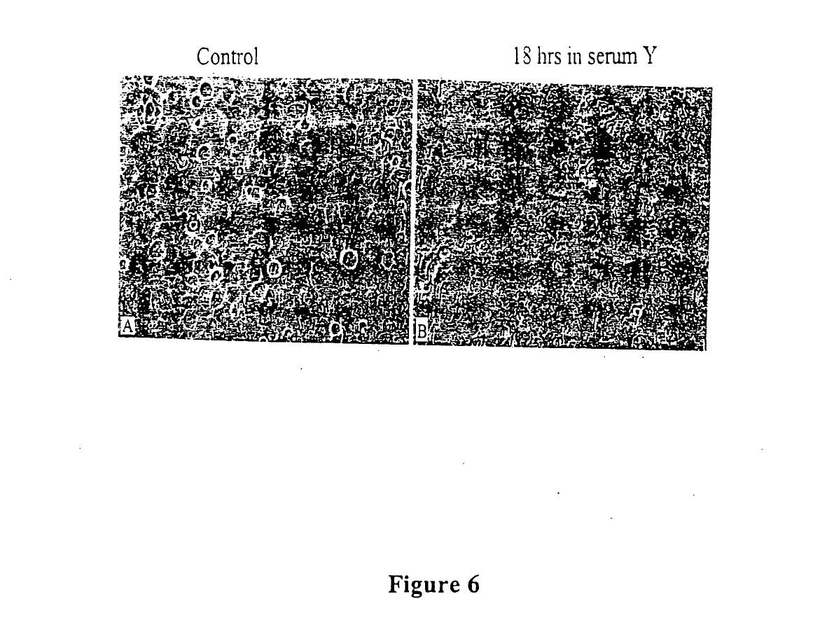

The effect of alligator serum on different human tumors is summarized in FIG. 4, demonstrating that alligator serum inhibited the control MTT activity by 80-90% in all four cell types. The anti-tumor effect was expressed as % inhibition of the control MTT activity as determined in the presence of fetal calf serum (FCS). Results are expressed as mean±SEM. FIGS. 5 and 6 depict two representative experiments illustrating the morphological destruction of T47D and HeLa cells by alligator serum, denoted Serum Y. The cultures were incubated for 18 hr. with alligator serum.

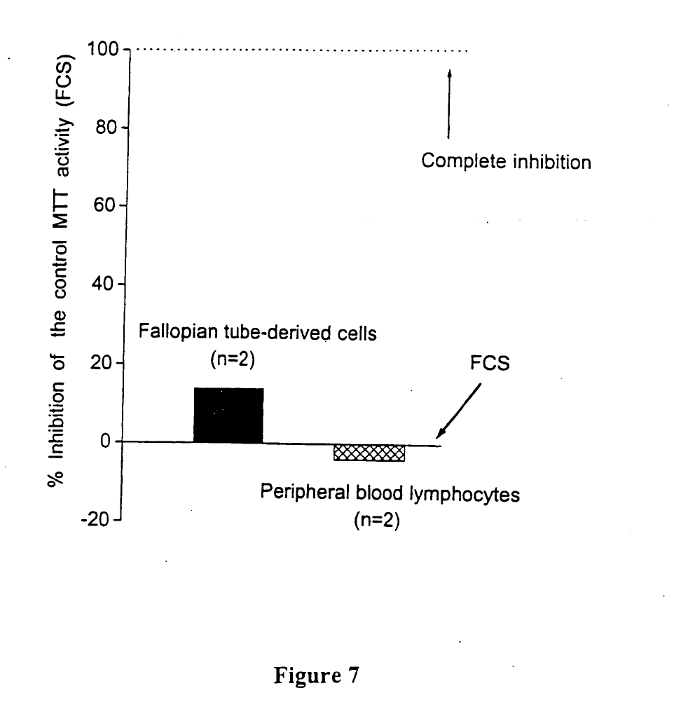

In contrast to the marked inhibitory effect of alligator serum on human tumor cells, in control experiments employing normal human cells (FIG. 7) we found that alligator serum at a dose twice that capable of killing human tumor cells, only slightly affected fallopian tube derived cells (FDTC), while MTT activity of peripheral blood lymphocytes was unaffected (the negative value indicates slight stimulation compared to control MTT activity in FCS). Cells were exposed to alligator serum for 18 hours prior to assaying the MTT activity. The results are expressed as % inhibition of the control MTT activity as determined in the presence of fetal calf serum (FCS).

Specificity of Anti-Tumor Activity of Alligator Serum as Compared with Cytosar

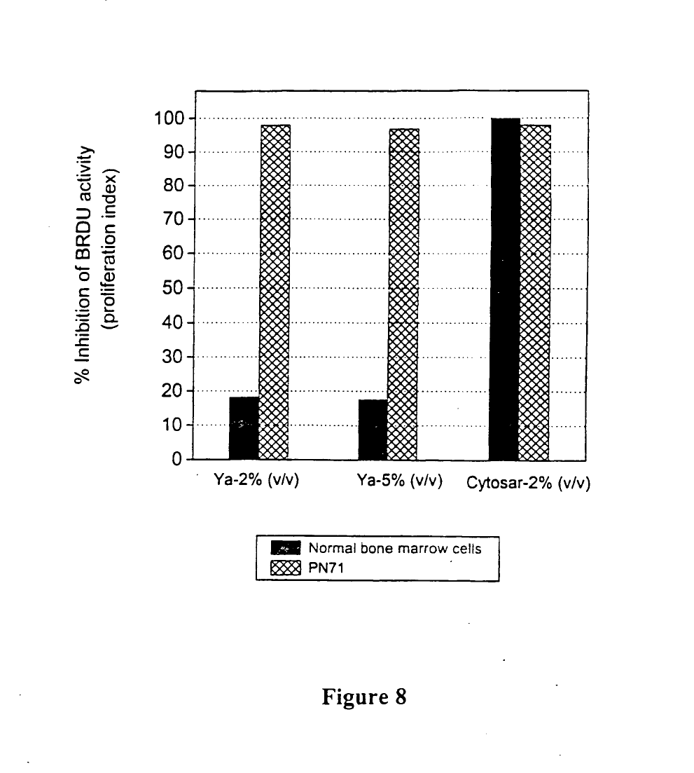

A remarkable feature of the anti-tumor activity herein reported, distinguishing it from common chemotherapeutic agents, is its ability to discriminate between normal proliferating cells and tumor (malignant) cells as described in FIG. 8. While fraction Ya (45% ammonium sulfate precipitatate) markedly (˜98%) inhibits PN71 cells, it is hardly affects normal bone marrow cells. In contrast, Cytosar (Cytarabine, Upjohn), commonly used in human chemotherapy, indiscriminately destroys both proliferating cell tapes.

Chemical Nature and Physical Properties of the Anti-Tumor Compound

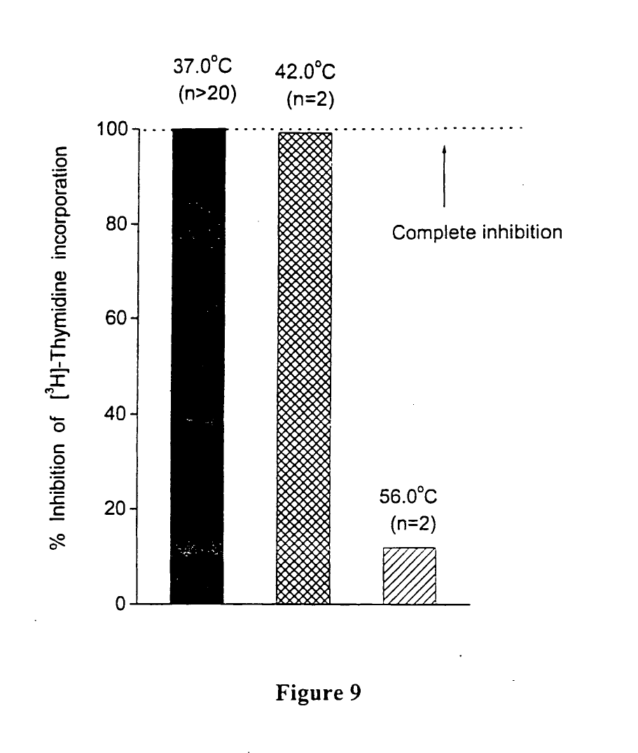

The heat sensitivity of the anti-tumor activity of alligator serum is illustrated in FIG. 9. Alligator serum was exposed to elevated temperatures and then evaluated for its anti-tumor activity. The strong attenuation of the biological activity at 56.0° C. suggests that the anti-tumor compound is a protein. Alligator serum was incubated for 30 min. at 42.0° C. and 56.0° C. before addition to the culture medium. Cells were exposed to alligator serum for 18 hours prior to adding [³H]-Thymidine. The anti-tumor effect was expressed as % inhibition of the control MTT activity as determined in the presence of fetal calf serum (FCS).

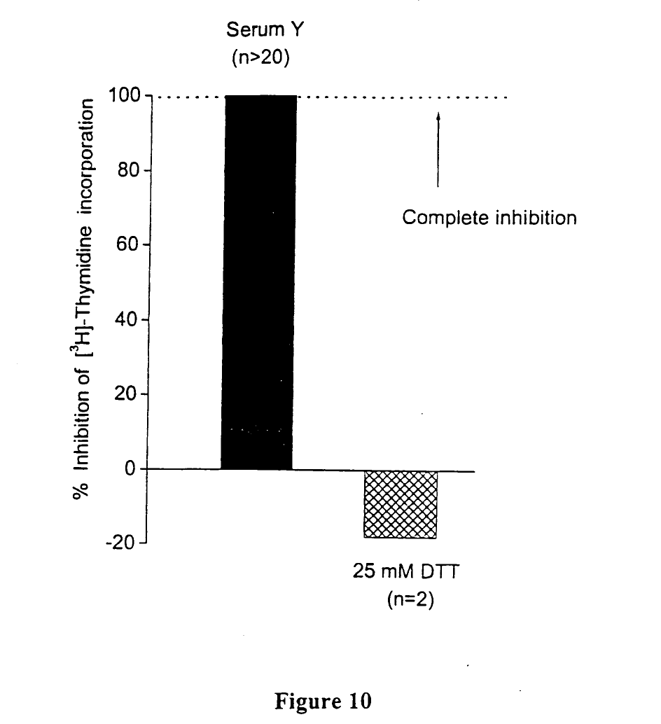

This notion is further supported by experiments demonstrating that dithiotreitiol (DTT) which reduces disulfide bonds, markedly inhibited the anti-tumor activity against PN71 cells (FIG. 10). Alligator serum was incubated with DTT for 2 hr. prior to addition of the serum to the culture medium. Cells were exposed to serum Y for 18 hours before adding [³H]-Thymidine. Inhibition of [³H]-Thymidine incorporation by untreated alligator serum was defined as 100%.

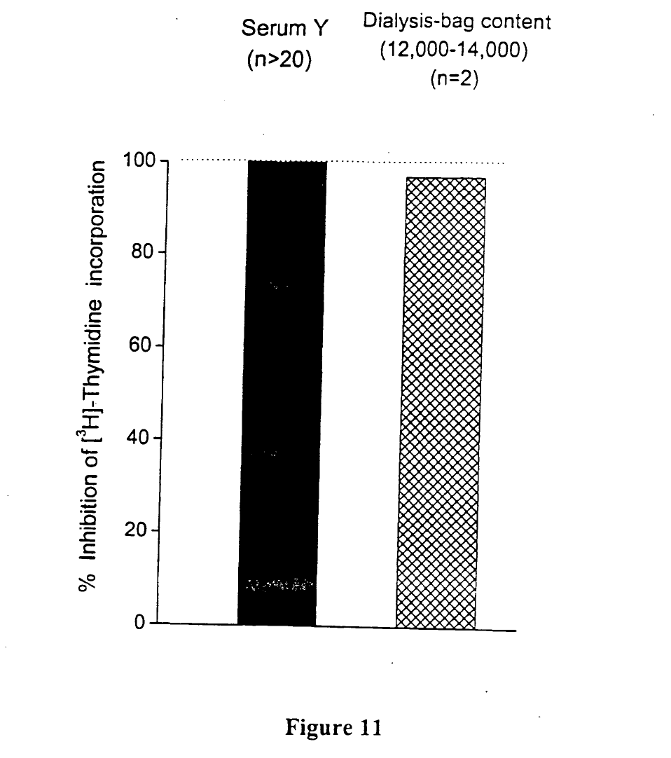

Finally, the results of the dialysis experiments shown in FIG. 11 indicate that the anti-tumor compound is of molecular weight greater than 12,000 Daltons. Cultures were exposed to alligator serum or to the retained contents of the dialysis-bag (12,000-14,000 bag cutoff) for 18 hours prior to addition of [³H]-Thymidine. Inhibition of [³H]-Thymidine incorporation by pre-dialysis alligator serum was defined as 100%.

Ammonium Sulfate Fractionation of Alligator Serum

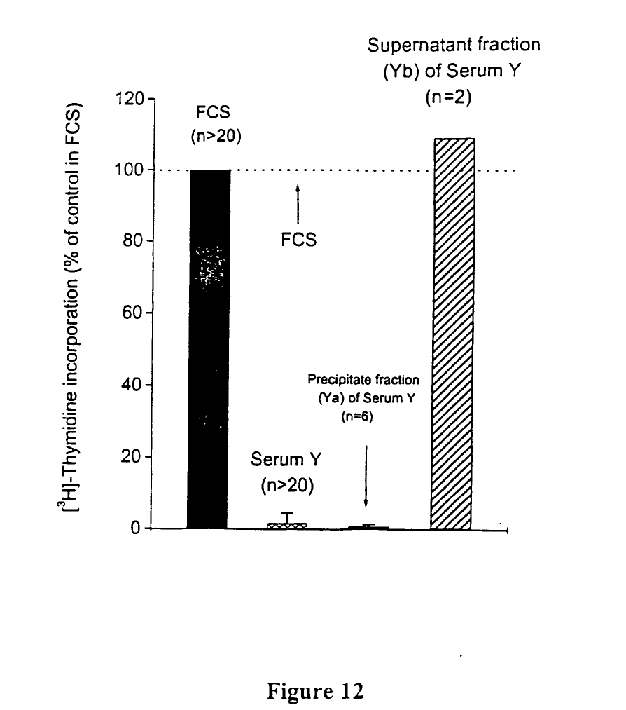

FIG. 12 depicts the anti-tumor activity of the precipitate (Ya) and the supernatant (Yb) components obtained by fractionation of alligator serum with 45% ammonium sulfate. It is seen that the entire biological activity was retained within the precipitate fraction (Ya), as demonstrated by the marked inhibition of [³H]-Thymidine incorporation of PN71 cells. [³H]-Thymidine incorporation in FCS was defined as 100%.

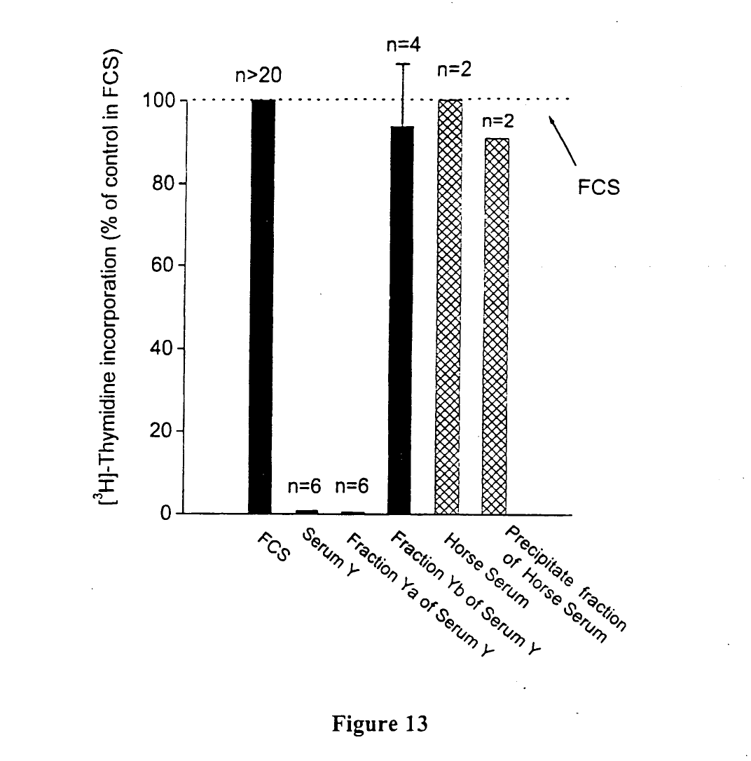

As depicted in FIG. 13, the anti-tumor activity against EL4 cells was also retained in fraction Ya. The [³H]-Thymidine incorporation in FCS was defined as 100%. Fractionation of horse serum was performed as a control in order to demonstrate that ammonium sulfate fractionation (fraction a) per se, did not generate any anti-tumor activity or inhibitory potency in horse serum.

Fractionation of Alligator Serum: MTT Assay

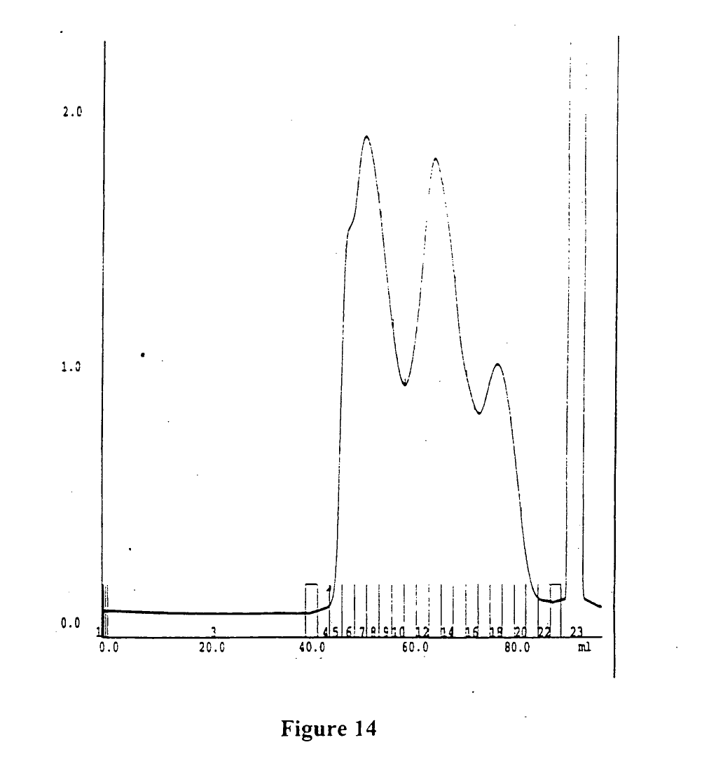

The 45% ammonium sulfate precipitate fraction was further fractionated on FPLC, using Sephacryl S-200 superfine column. FIG. 14 depicts the protein content distribution throughout the column.

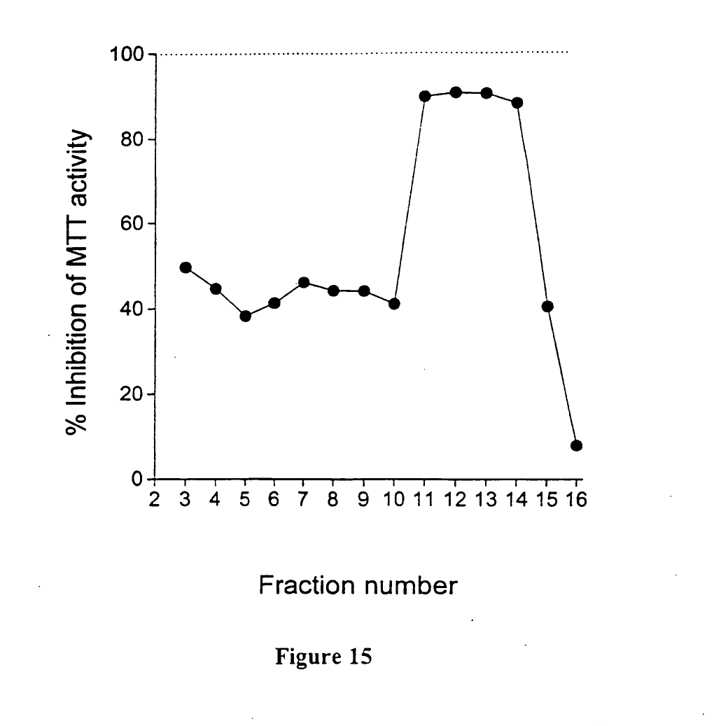

Most of the proteins were eluted in fractions 3-20 (FIG. 14). Anti-tumor activity was determined by the MTT assay (FIG. 15). The anti-tumor effect was expressed as % inhibition of the control MTT activity, as determined in the presence of fetal calf serum (FCS). It is seen that fractions 11-14 caused over 90% inhibition of the control (in FCS) MTT activity.

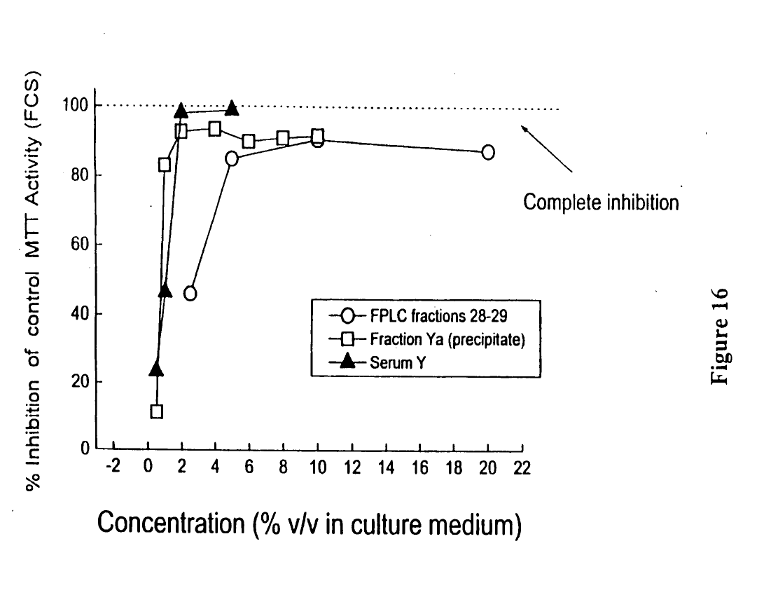

FIG. 16 depicts the dose-response relations of fractions 12-13 (shown in FIG. 14) and fraction Ya (the crude precipitate). The anti-tumor activity against PN71 cells was expressed as % inhibition of the control MTT activity, as determined in the presence of fetal calf serum (FCS). The cells (2.5×10⁴ PN71 CTL hybridoma) were incubated for 18 hr. with each fraction.

Table 1 below demonstrates the increase in the specific activity of the anti-tumor compound by the purification process of the crude material. The specific activity is expressed as unit/mg protein. A unit is defined as the amount that causes 50% reduction of the MT activity (compared to control in FCS, which is defined as 100% MTT activity), of PN71 cells, after incubation for 18 hr. with alligator serum. The data in following table were obtained by a representative experiment.

TABLE 1
Specific activity of anti-tumor preparations

| | | Specific activity |
|---|---|---|
| Fraction | Label | (units/mg protein) |
| Crude material | Serum Y | 16.6 |
| Precipitate after fractionation | fraction Ya | 41.6 |
| with 45% ammonium sulfate | | |
| FPLC fractionation-S200 col- | fractions #12-13 | 108.0 |
| umn | | |

The results depicted in FIGS. 14-16 represent over 30 fractionation experiments with similar results.

FPLC (S-200) Fractionation-SDS PAGE

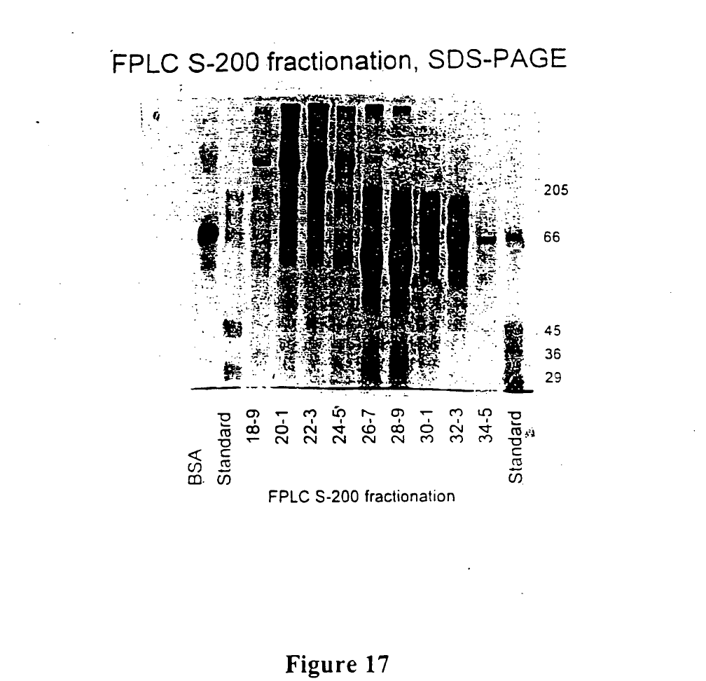

FIG. 17 demonstrates SDS-PAGE of nine combined fractions (3-20) eluted from FPLC column (see FIG. 14). Electrophoresis under denaturing conditions of fractions 11-12 and 13-14 which contain the anti-tumor activity, resulted in two major bands, one of approximately 60 kD and the second of approximately 30 kD, in each of these samples. The molecular weight of the intact native protein eluted in these fractions (# 11-14), is approximately 150 kD, and is distributed into two subunits, resembling the commonly occurring IgG heavy and light chains.

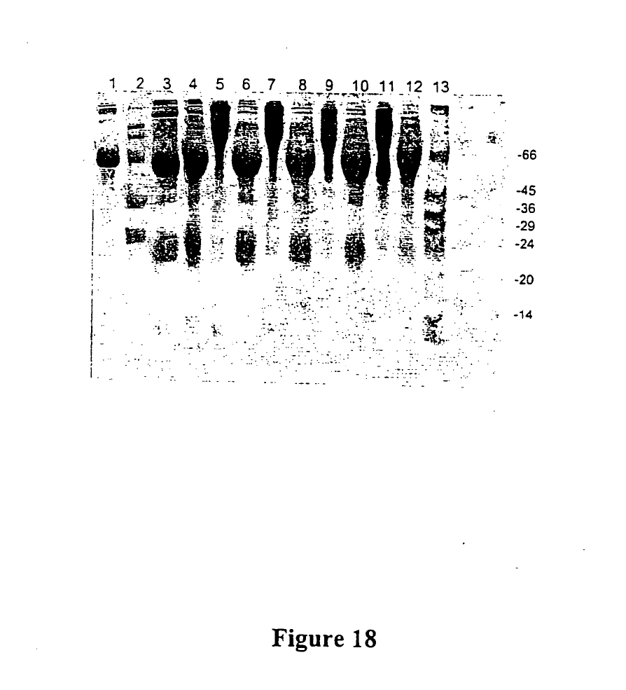

As mentioned above, the fractions that contain the anti-tumor activity include proteins of a molecular mass of about 150 kDa which correspond to the molecular mass of IgG. As shown in FIG. 18, SDS-PAGE analysis of denatured proteins from these fractions, revealed two major bands at about 60 and about 30 kDa, corresponding to the heavy and light chains of IgG, respectively. The non-denatured proteins migrated at about 150 kDa, corresponding to the intact IgG molecule. While it is possible that the anti-tumor protein is an IgG molecule, this is not necessary the case since other proteins may have the same molecular weight values under denaturing and non-denaturing conditions.

Synergistic Effect of Two Distinct Activities Separated by Superdex S-200 Column

Fractionation of Ya (45% ammonium sulfate precipitate) in FPLC (S-200) shown in FIG. 14, resulted in a major peak of anti-tumor activity at fractions 11-14 (Fraction AF2), and a small peak induced by Fractions 4-8 (Fraction AF1). Exposure of tumor cell cultures to these isolated fractions showed that the effects of these fractions were distinct.

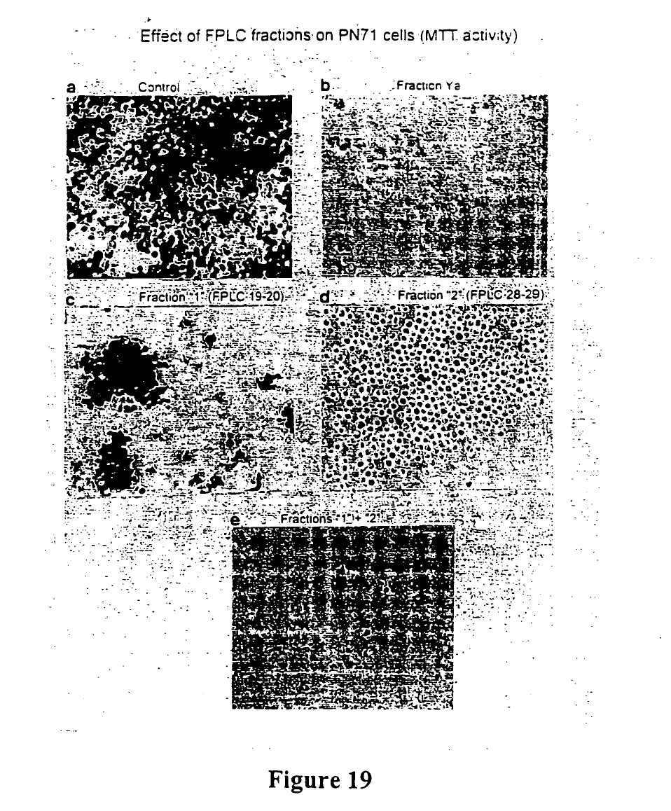

FIG. 19 depicts the effects of AF1 and AF2 on the morphological appearance of PN71 cultures. 2.5×10⁴ cells were cultured for 18 hr. in the presence 5% (v/v) of: a: Fetal calf serum (FCS, control); b: Ya (45% ammonium sulfate fractionation); c: AF1 (Fractions 5-6 from FPLC fractionation); d: AF2 (Fractions 12-13); e: AF1+AF2. In the control culture (panel a), there is a large population of intensively MTT-stained cells, indicating intact and functional cells. The Ya-treated culture (b) contains aggregates of lyzed, MTT-negative cells, indicating cell destruction. AF1 (c), caused aggregation of intact, MTT-positive cells. Treatment with AF2 (d), resulted in dispersed and lyzed, MTT-negative cells. Treatment with AF1+AF2 (e) induced an effect similar to that of Fraction Ya (b), namely, aggregates of lyzed, MTT-negative cells.

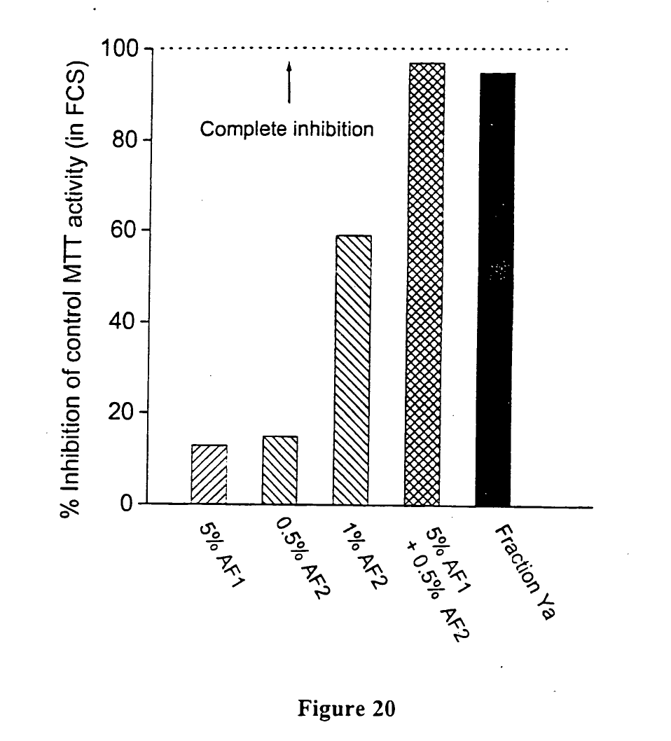

FIG. 20 depicts the synergistic effect of AF1 and AF2 on the MTT activity of PN71 cells. The figure shows the effects of 5% (v/v) Fraction 1, 0.5 and 1% Fraction 2, and the combination of 5% Fraction 1 and 0.5% Fraction 2, on the MTT activity of PN71 cells. 5% (v/v) of Fraction 1 caused 13% inhibition of the control MTT activity. 0.5% Fraction 2 caused 15% inhibition of the control MTT activity. Combination of both Fractions inhibited the control MTT activity by approximately 98%. Most importantly, this marked inhibition is much higher than the sum of inhibitions caused by each Fraction administered separately.

The major technique currently employed to purify the anti-tumor protein is FPLC gel filtration fractionation, which enables to identify the fractions exhibiting the anti-tumor activity. The distribution of the FPLC fractionation is composed of two distinct components: (i) anti-tumor activity that reaches a maximum of 40% is resolved in fractions 3-10; and (ii) anti-tumor activity that reaches a maximum of >90% is resolved in fractions 11-14.

In order to calculate the combined ‘recovered’ anti-tumor activity of fractions 11-14 (the “output”) which include the main anti-tumor activity, as compared with that of the crude fraction Ya (the “input”), as shown in FIG. 20, dose-response relations of fraction Ya and of fractions 11-14 were determined using the MTT activity inhibition assay.

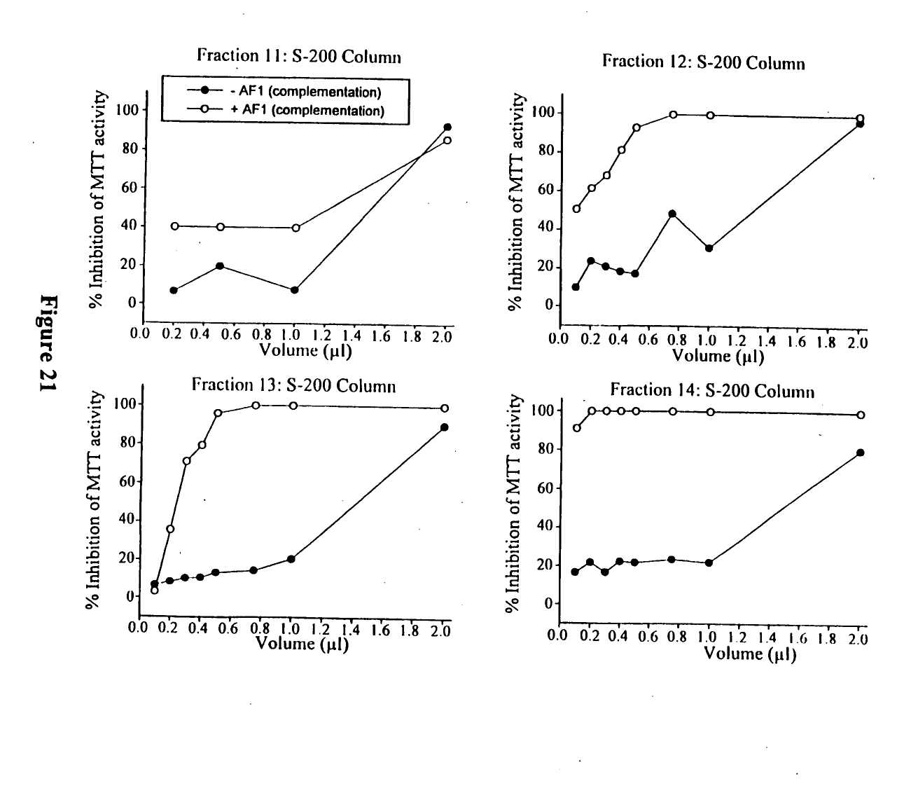

From these curves, the anti-tumor activity was calculated as follows: as mentioned above, one unit of anti-tumor activity is defined as the volume causing 50% inhibition (of MTT activity) when tested on 2×10⁴ PN71 cells. In FIG. 16 this volume is 0.3 μl. As 1,800 μl of fraction Ya were loaded on the FPLC column, the total activity of fraction Ya was 6,000 units. Based on the dose-response curves shown in FIG. 21, the resulting combined anti-tumor activity of fractions 11-14 is 960 units, 16% of the total activity present in fraction Ya.

Due to the low (about 16%) recovery of the anti-tumor activity of fractions 11-14 and based on the observations of morphological complementation described in FIG. 19, we examined the hypothesis that the low yield of anti-tumor activity recovered after the FPLC fractionation is caused by the separation of complementary (anti-tumor) activity that is resolved in fractions 3-10 from the main anti-tumor protein residing in fractions 11-14.

To assess complementation quantitatively, dose-response curves for fractions 11-14 were generated in the absence and presence of 2 μl of fractions 5-6 (FIG. 21). It is clearly seen that addition of the “aggregating molecule” (AF1) to each of the fractions, shifted the original dose-response curves to the left and upwards. From these curves, the (calculated) combined anti-tumor activity of the ‘complemented’ fractions was calculated to be 6,366 units.

Thus, it is clear that Serum Y contains at least two factors that act in concert in order to exert the tumor cell killing effect. One factor has a molecular weight of about 150 kDa while the other is of about 700 kDa.

The possibility that the aggregating factor (700 kDa) specifically, recognizes tumor cells, but not normal cells was investigated. For non-tumor control cell NHM cells obtained from two normal subjects were used. Incubation of NHM cells with fractions 5+6 (the aggregating fractions) caused neither aggregation nor killing of these cells, suggesting that the aggregating protein has a specific affinity towards tumor cells.

Lack of Toxicity of Alligator Serum in Mice

The experimental points addressed were as follows: 1) whether alligator serum induces acute toxicity, and 2) will repeated injections of alligator serum initiate an adverse or lethal antigenic response.

ICR strain mice, weighing 13-15 gr. were injected intraperitoneally with 300 μl (n=3) or with 400 μl (n=1), of the precipitate fraction product of the crude material obtained after fractionation with 45% ammonium sulfate. Assuming a blood volume of ˜1.5 ml (10% of body weight), 300 μl of active fraction in 1.5 ml blood, would yield a concentration of 20%. Please note that 0.5-2% of crude material caused complete inhibition of cultured tumor cells. Before administration to mice, the fraction was filtered through a 0.45 μm filter, which did not affect the anti-tumor potency, as assayed in cell cultures. The first intraperitoneal administration was followed by a second administration (using the same doses) on day # 2. No overt toxicity was observed, as judged by inspecting the mice in their cages following two administrations of alligator serum. Following these administrations, the mice were observed daily for 20 days, and exhibited no abnormal behavior or gross pathology. On the 20th day following the second injection, mice (n=4) were injected with an additional dose (400 μl) of the active fraction. No visible effects were observed in the mice, in response to administration of the ‘challenge dose’ of the active fraction. These studies provide an important indication that no serious adverse or lethal antigenic response has developed by repetitive administrations of alligator serum.

Monoclonal Antibodies Against the Small Anti-Tumor Molecule

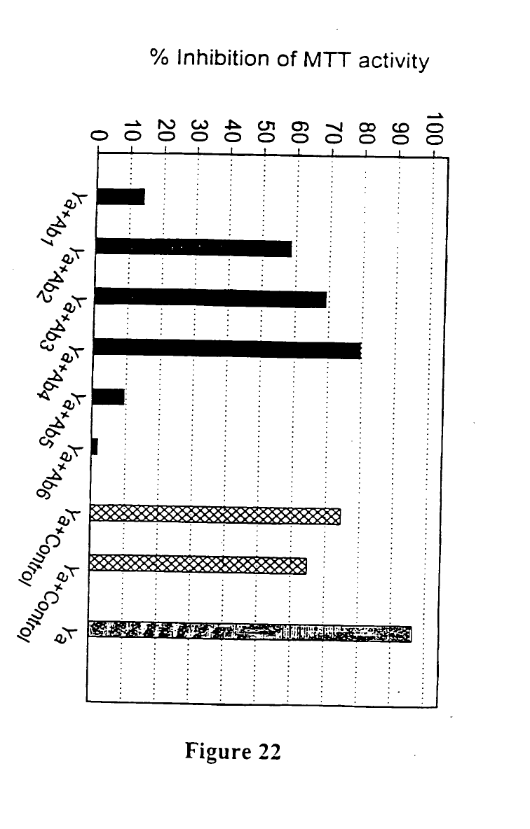

As illustrated in FIG. 22 three out of six sera from immunized mice (Ab1, Ab5 and Ab6) contained antibodies that markedly neutralized the anti-rumor activity of fraction Ya as tested by monitoring MTT activity in PN71 cells.

Characterization of class and subclass of the mAb was carried out using the ELISA assay. Most clones produced γ1/K mAb; clones producing other classes of mAb were excluded.

Quantification of IgG mAb in the supernatant produced by the positive clones was performed using a standard curve generated for IgG doses and detected using an ELISA assay.

Biological activity of the clones was determined in the same supernatant media used for the quantification. Media were incubated (at different dilutions) with fraction Ya prior to exposure of PN71 cells to Ya. PN71 cell viability, represented by the MTT activity, was increased with progressively higher mAb concentration. Namely, larger concentrations of neutralizing mAb caused augmented inhibition of the anti-tumor efficacy of fraction Ya, resulting in higher survival of tumor cells.

The actual MET value corresponding to 1 biological unit of mAb (as defined above) was calculated for each dose-response curve of biological activity of the clone-derived supernatant. This value was determined based on the MTT activity of the fetal calf serum (FCS)-treated PN71 cells, defined as 100% activity, and the MTT value of Ya-treated cells, defined as the maximal inhibition value (expressed as percentage of the FCS-treated value).

One unit of mAb activity was defined as the amount of the clone's supernatant resulting in MTT activity corresponding to 40% inhibition of the maximal inhibition value compared to the maximal MTT of FCS-treated cells (namely neutralization of 60% of the killing capacity of Ya under the assay conditions). The actual amount of supernatant yielding this value of MTT activity (e.g., 1 unit) was determined from the dose-response curve generated for each clone.

Table 2 summarizes the characterization of 4 representative active clones. Each clone was screened for its typing, class and subclass, its biological activity (i.e., the ability to block Ya anti-tumor activity), and IgG concentration. The mAb concentration and its biological activity were used to calculate the specific activity of each clone.

Clones producing non-relevant mAb were used as negative controls. Supernatant (from these clones) containing the same IgG class, and IgG concentrations similar to those used for screening the anti-Ya mAb producing clones, were used in the “neutralization assay”. None of the supernatants from the non-relevant clones had any neutralizing effect on Ya anti-tumor activity.

Clones with high (>10 units/ng IgG) specific activities were frozen and kept at −70° C. These active clones, which produce the anti-tumor neutralizing mAb, are used to characterize the active anti-tumor protein by means of Western blot analysis, as well as for purification of the active molecule using the affinity chromatography technique.

TABLE 2
Characterization of four positive mAb clones (>60% Neutralization of
Ya Activity)

| Cloning | | | | | Specific activity |
|---|---|---|---|---|---|
| origin | Clone # | Type | Units/ml | ng Ab/ml | (units/ng ab) |
| 6/011 | 12/D3+ | γ1/k | 61.0 | 4.34 | 14.1 |
| 14/H4 | 25/F5 | γ1/k | 15.4 | 1.80 | 8.6 |
| 4H/1 | 25/C2+ | γ1/k | 73.2 | 1.80 | 40.7 |
| 4H/1 | 26/C5 | γ1/k | 15.7 | 1.49 | 10.5 |

These antibodies will be used for purification and characterization of the small anti-tumor molecule. Purification will be achieved using the mAb, combined with affinity chromatography techniques. Subsequently, a comprehensive physical and biochemical characterization of the small anti-tumor protein will be performed. Once the anti-tumor protein is at hand, it will be fragmented by common techniques, thus exploring the ability of smaller components to maintain the anti-tumor activity.

Purification and Characterization of the Large Anti-Tumor Molecule

To purify the large anti-tumor (aggregating) molecule, two distinct and independent experimental approaches will be used:

(1) Purification via “classical” biochemical approach, such as gel filtration, ion exchange, hydrophobic, heparin, and other types of chromatography. In each step, the different column fractions will be assayed for anti-tumor activity (death of cells; MTT level) against tumor cultured cells as described in the patent application and preliminary results.

(2) Development of a monoclonal antibody (mAb) against the active protein. As performed for the small anti-tumor molecule. The anti-tumor protein will be purified via affinity chromatography over the appropriate inhibitory antibody that will be immobilized.

In Vivo Studies

Murine tumors are selected based on their clinical relevance to human tumors. These studies will be initiated by exploring the in vivo efficacy of the anti-tumor molecules in murine peritoneal leukemia.

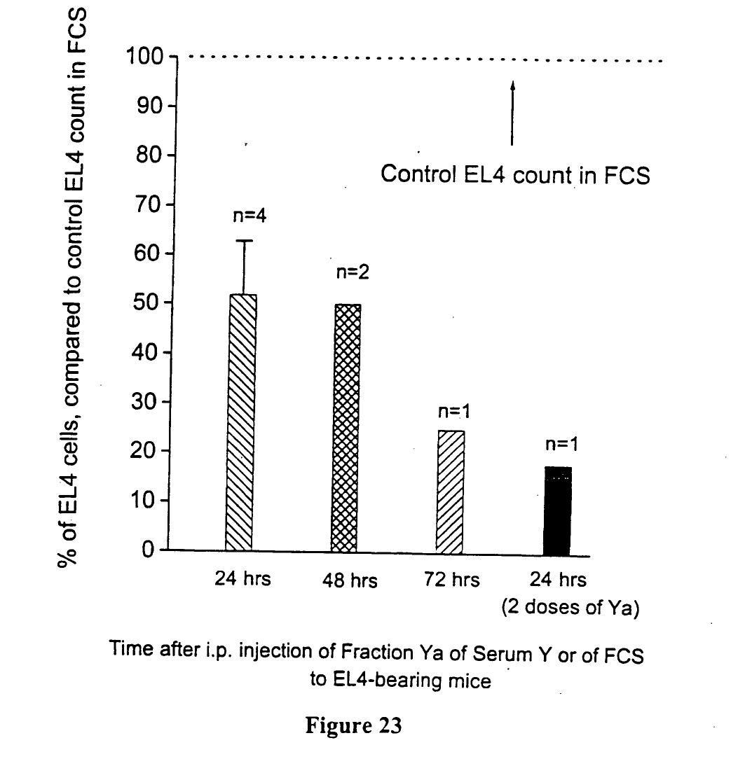

In vivo anti-tumor activity of alligator serum was tested in leukemic mice. The murine syngeneic peritoneal EL4 leukemia model was established by injecting 0.5×10⁶ cells intraperitoneally (i.p.) into inbred strain C57B1/6 mice. Fraction Ya (45% ammonium sulfate precipitation) of alligator serum or of FCS (control) was injected intraperitoneally 48 hr. after EL4 administration, when the leukemic cell count was approximately 5-8×10⁶. Fraction Ya was injected at a dose of 100 μL/10⁶ EL4 cells. Animals were sacrificed 24, 48, and 72 hr. after Ya administration, and the peritoneal EL4 cell count was determined. In the experiments depicted in FIG. 23, the first three bars represent the inhibition of tumor cell growth by fraction Ya of alligator serum relative to the control, at the different time points tested. In the experiment represented by the black bar of FIG. 23, the mouse was injected i.p. with two consecutive doses, 6 hr. apart, of Fraction Ya, beginning 24 hr. after i.p. inoculation with 0.5×10⁶ EL4 cells. The mouse was sacrificed 24 hr. after the second dose and peritoneal EL4 cell count was determined to be 82% inhibited compared to the control. Hence, in all experiments pronounced inhibition of tumor cell proliferation was noted.

Additional more clinically relevant in vivo models will test the effect of the anti-tumor agents on human tumors transplanted in nude mice.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention. Thus the expressions “means to . . . ” and “means for . . . ”, or any method step language, as may be found in the specification above and/or in the claims below, followed by a functional statement, are intended to define and cover whatever chemical structure, or whatever function, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

What is claimed is:

1. An anti-tumor agent derived from reptile serum, comprising at least one serum protein from normal reptile serum.

2. The anti-tumor agent of claim 1 wherein the serum protein is obtained from normal alligator serum.

3. The anti-tumor agent of claim 1 wherein at least one serum protein has a molecular weight in excess of 100,000 Daltons in its native form.

4. The anti-tumor agent of claim 3 wherein at least one serum protein has a molecular weight of about 150,000 Daltons in its native form.

5. The anti-tumor agent of claim 1 wherein the anti-tumor agent comprises at least two proteins.

6. The anti-tumor agent of claim 5 wherein the anti-tumor agent comprises a first protein having a molecular weight in excess of 100,000 Daltons and a second protein having a molecular weight in excess of 200,000 Daltons.

7. The anti-tumor agent of claim 5 wherein the anti-tumor agent comprises a first protein having a molecular weight of about 150,000 Daltons and a second protein having a molecular weight of about 700,000 Daltons.

8. The anti-tumor agent of claim 4 wherein the protein is an immunoglobulin.

9. The anti-tumor agent of claim 1 wherein the protein is in essentially isolated form.

10. An anti-tumor agent derived from reptile serum comprising a subunit of an anti-tumor protein derived from reptile serum.

11. An anti-tumor agent derived from reptile serum comprising a fragment of an anti tumor protein derived from reptile serum which retains the activity of the intact protein.

12. An anti-tumor agent derived from reptile serum comprising an active fragment of an immunoglobulin molecule according to claim 8.

13. An anti-tumor agent derived from reptile serum comprising an active peptide derived from an anti-tumor protein.

14. A pharmaceutical composition comprising as an active ingredient the anti-tumor agent of claim 1.

15. A pharmaceutical composition comprising as an active ingredient the anti-tumor agent of claim 11.

16. A pharmaceutical composition comprising as an active ingredient the anti-tumor agent of claim 12.

17. A pharmaceutical composition comprising as an active ingredient the anti-tumor agent of claim 13.

18. A diagnostic reagent comprising as an active ingredient the anti-tumor agent of claim 1.

19. A diagnostic reagent comprising as an active ingredient the anti-tumor agent of claim 11.

20. A diagnostic reagent comprising as an active ingredient the anti-tumor agent of claim 12.

21. A diagnostic reagent comprising as an active ingredient the anti-tumor agent of claim 13.

22. A process for preparing an anti-tumor agent from reptile serum comprising the steps of: a) fractionating reptile serum by adding an ammonium sulfate salt in an amount of about 45% of the amount necessary to form a saturated solution; b) centrifuging the serum to recover the precipitated proteins; c) redissolving the precipitate and desalting the recovered proteins; d) gel filtering the desalted proteins; and e) collecting an active fraction.

23. The process according to claim 22 wherein the active fraction comprises a protein having a molecular weight of approximately 150,000 Daltons.

24. The process according to claim 23 further comprising isolating the protein in essentially purified form.

25. The process according to claim 24 wherein the protein is affinity purified using a specific antibody.

26. An anti-tumor agent from reptile serum comprising at least one protein from the serum of normal alligators obtained by a process comprising the steps of: a) fractionating the serum by the addition of ammonium sulfate salt in an amount of about 45% of the amount necessary to form a saturated solution; b) centrifuging the serum to recover the precipitated proteins; c) redissolving the precipitate and desalting the recovered proteins; d) gel filtering the desalted proteins; and e) collecting at least one active fraction.

27. The anti-tumor agent of claim 26 wherein the anti-tumor agent comprises at least two serum proteins.

28. A method of treating or preventing cancer which comprises administering to a mammal in need thereof a pharmaceutical composition comprising as an active ingredient a therapeutically effective amount of an anti-tumor agent derived from reptile serum and comprising at least one serum protein found in normal reptiles.

29. A method of treating or preventing cancer comprising administering to a mammal in need thereof a pharmaceutical composition comprising as an active ingredient a therapeutically effective amount of an anti-tumor agent derived from the serum of reptiles comprising at least one active fragment of a serum protein found in normal reptiles.

30. A method of treating cancer comprising administering to a mammal in need thereof a pharmaceutical composition comprising as an active ingredient a therapeutically effective amount of an anti-tumor agent derived from reptile serum and comprising at least one active fragment of a serum protein found in normal reptiles and being covalently coupled to an anticancer drug.

31. A method of diagnosing or imaging cancer comprising utilizing a diagnostic reagent comprising as an active ingredient an anti-tumor agent derived from reptile serum and comprising at least one serum protein found in normal reptiles.

32. A method of diagnosing or imaging cancer comprising utilizing a diagnostic reagent comprising as an active ingredient an anti-tumor agent derived from reptile serum and comprising at least one active fragment of a serum protein found in normal reptiles.

33. A method of diagnosing or imaging cancer comprising administering to a mammal a diagnostic reagent comprising as an active ingredient an anti-tumor agent derived from reptile serum comprising at least one serum protein found in normal reptiles.

34. A method of diagnosing or imaging cancer comprising administering to a mammal a diagnostic reagent comprising as an active ingredient an anti-tumor agent derived from reptile serum and comprising at least one active fragment of a serum protein found in normal reptiles.

35. An antibody preparation comprising at least one antibody recognizing an anti-tumor agent derived from reptile serum.
